(19) United States
(12) Patent Application Publication
(10) Pub. No.: US 2026/0096920 A1
(43) Pub. Date: Apr. 9, 2026

(54) THERMOTHERAPY DEVICE FOR PET USE AND METHOD OF USING SAME

(71) Applicant: The PetCot Company, O'Fallon, MO (US)

(72) Inventor: Greg Squires, O'Fallon, MO (US)

(21) Appl. No.: 19/415,334

(22) Filed: Dec. 10, 2025

Related U.S. Application Data
(60) Provisional application No. 63/781,671, filed on Apr. 1, 2025.
(60) Provisional application No. 63/819,272, filed on Jun. 6, 2025.

(51) Int. Cl. A61F 7/00; A01K 1/00; A01K 1/035

(52) U.S. Cl. CPC A61F 7/0053; A01K 1/0064; A01K 1/0353; A61F 2007/0055

(57) ABSTRACT

A thermotherapy device for use in a kennel includes a ducted pet bed inside the kennel. A support sheet supports a pet and a plenum is below the support sheet. The support sheet defines a vent in fluid communication with the plenum. An interior air fitting is supported on the ducted pet bed in fluid communication with the plenum and positioned adjacent a doorway to oppose an interior side of a door when closed. The interior air fitting makes a fluid connection to the door when closed so that temperature controlled air flows through door openings into the plenum, then passes through the vent toward the pet. The device may include an exterior air fitting mounted on the door's exterior side that aligns with the interior air fitting when closed.

### CROSS-REFERENCE TO RELATED APPLICATIONS

This application claims priority U.S. Provisional Patent Application No. 63/781,671, titled THERMOTHERAPY DEVICE FOR PET USE AND METHOD OF USING SAME, filed Apr. 1, 2025, and No. 63/819,272, titled THERMOTHERAPY DEVICE FOR PET USE AND METHOD OF USING SAME, filed Jun. 6, 2025, each of which is hereby incorporated by reference in its entirety.

### FIELD

The present disclosure relates generally to temperature control systems for pets, in particular, to a system that uses a ducted pet bed to warm or cool a pet, typically after a veterinary procedure.

### BACKGROUND

In veterinary medicine, thermotherapy devices are used to maintain a patient's temperature during treatment and healing. For example, thermotherapy devices are commonly employed to keep patients warm in a kennel during post-surgery recovery. A conventional thermotherapy device comprises a blanket arrangement from at least two layers to create a hollow space for air delivery. The blanket may have air holes or be made of a porous material to allow warm air to reach the patient. The thermotherapy blanket connects to a forced air heating unit that generates warm air. Thermotherapy blankets were previously used to keep humans warm in the medical context and were later adapted to veterinary applications.

But using a thermotherapy blanket in a veterinary application inside a kennel presents challenges. The heating unit must be outside the kennel, while the blanket is inside, making it difficult to deliver the warm air from the heater to the patient. The typical solution is to mount a two-part fitting on opposite sides of the barred kennel door. Each fitting part includes a tube with a flange at one end. The flange of one fitting part is positioned against the interior side of the kennel door, the flange of the other fitting part is positioned against the exterior side of the kennel door so that the tubes of the two fitting parts are substantially aligned, and the two flanges are secured together. An upstream hose runs from the exterior side fitting part to the heating unit, and a downstream hose runs from the interior side fitting part to the inlet of the blanket.

### SUMMARY

Some exemplary embodiments are related to a thermotherapy device for use in a kennel. The thermotherapy device comprises a ducted pet bed sized and arranged for being positioned inside the kennel, the ducted pet bed comprising a support sheet for supporting a pet and a plenum below the support sheet, the support sheet defining a vent in fluid communication with the plenum through which air moving in the plenum is passable toward the pet supported on the support sheet. The thermotherapy device comprises an interior air fitting supported on the ducted pet bed in fluid communication with the plenum. The ducted pet bed and the interior air fitting are arranged so that, when the ducted pet bed is positioned inside the kennel the interior air fitting is located adjacent a doorway of the kennel to oppose an interior side of a door of the kennel when the door is in a closed position and to be separated from the door when the door moves away from the doorway to an open position. The interior air fitting is configured to make a fluid connection to the door when the door is in the closed position so that the interior air fitting passes temperature controlled air from a temperature control device flowing through one or more openings in the door into the plenum, whereby the temperature controlled air is caused to move into the plenum and pass through the vent toward the pet supported on the support sheet.

Other exemplary embodiments are related to a thermotherapy device for a kennel. The thermotherapy device comprises a cot comprising a frame assembly including legs and a perimeter frame supported on the legs, the legs configured to support the perimeter frame at an elevated position. The cot comprises a support sheet stretched across the perimeter frame at the elevated position for supporting a pet at the elevated position, the support sheet defining a vent through which air is passable across the support sheet. The cot comprises an interior air fitting connected to the frame assembly, the interior air fitting comprising an inlet and an outlet. The cot comprises a ducting panel below the support sheet, the ducting panel engaged with the support sheet and the interior air fitting such that the ducting panel defines a plenum connecting the outlet of the interior air fitting to the vent such that air forced into the air fitting through the inlet flows from the outlet into the plenum and the plenum guides the forced air to flow through the vent to the pet supported on the support sheet.

Still further exemplary embodiments are related to a method of treating a pet in a kennel. The method comprises deploying a ducted pet bed and an interior air fitting in the kennel such that the interior air fitting has an open inlet end adjacent a doorway of the kennel. The method comprises bringing the pet into the kennel through the doorway to rest on the ducted pet bed while a door of the kennel is in an open position such that an exterior air fitting supported on the door is fluidly disconnected from the interior air fitting. The method comprises closing a door of the kennel to fluidly connect an exterior air fitting supported on the door to the interior air fitting through one or more openings in the door. The method comprises activating a temperature control device to force temperature controlled air through the exterior air fitting into the interior air fitting, then from the interior air fitting into a plenum of the ducted pet bed, and from the plenum through a vent of the ducted pet bed.

Further exemplary embodiments are related to a method of treating a pet in a kennel. The method comprises deploying a pet bed having a pet support sheet elevated above an air flow space in the kennel. The method comprises bringing the pet into the kennel through the doorway to rest on the pet bed while a door of the kennel is open. The method comprises closing the door of the kennel to fluidly connect the exterior air fitting supported on the door to the air flow space. The method comprises activating a temperature control device to force temperature controlled air through the exterior air fitting into the air flow space and from the air flow space upward through a vent of the pet support sheet so that the temperature controlled air is directed onto the pet in the kennel from below.

Additional exemplary embodiments are related to a covering panel for use with a thermotherapy pet bed comprising a frame assembly holding a pet support surface above an air flow space. The covering panel comprises a topper portion configured to cover a support sheet of the pet bed, wherein the topper portion comprises air-permeable material configured to allow air from the air flow space to pass upward through the topper portion. The covering panel comprises a perimeter portion configured to extend around a perimeter of the frame assembly. The covering panel is configured to be installed on the thermotherapy pet bed while the thermotherapy pet bed is in a veterinary kennel having an exterior air flow fitting mounted to an exterior side of a door of the veterinary kennel. The covering panel is shaped and arranged to permit a fluid communication to be automatically made from the exterior air flow fitting to the air flow space when the door of the veterinary kennel is closed such that temperature controlled air imparted through the exterior air fitting is directed into the air flow space and from the air flow space upward through the pet support surface and through the topper portion toward a pet lying on the topper portion in the kennel.

Yet further exemplary embodiments are related to a veterinary kennel equipped for in-kennel thermotherapy. The veterinary kennel comprises a floor, walls extending upward from the floor, and a door movable between an open position and a closed position. The veterinary kennel comprises an exterior air fitting mounted on the door and configured to receive temperature controlled air from a temperature control device. The veterinary kennel comprises a pet bed positioned in the veterinary kennel, the pet bed comprising a support sheet elevated above the floor with an air flow space between the support sheet and the floor. The pet bed and the exterior air fitting are configured so that when the door is in the open position the exterior air fitting is fluidly disconnected from the air flow space and when the door is in the closed position the exterior air fitting is automatically fluidly connected to the air flow space such that temperature controlled air received by the exterior air fitting is directed into the air flow space and from the air flow space upward through the support sheet toward a pet supported on the support sheet.

Other aspects, embodiments, and features will be apparent hereinafter.

### BRIEF DESCRIPTION OF THE DRAWINGS

FIG. 1 is a schematic illustration of a thermotherapy device outside a kennel showing the door of the kennel separated from the main body of the kennel;

FIG. 2 is a schematic plan view of the thermotherapy device installed in and on the kennel with the kennel door closed;

FIG. 3 is a schematic plan view similar to FIG. 2 but showing the kennel door open;

FIG. 4 is a perspective of an embodiment of the thermotherapy device;

FIG. 5 is an exploded perspective of the thermotherapy device;

FIG. 6 is an exploded perspective of an interior air fitting of the thermotherapy device;

FIG. 7 is a fragmentary cross section of a portion of the thermotherapy device taken in a vertical plane along an axis of flow through the interior air fitting;

FIG. 8 is a cross section of the thermotherapy device taken in a vertical plane perpendicular to the plane of the cross section in FIG. 7 and looking away from the interior air fitting;

FIG. 9 is a perspective of an embodiment of the thermotherapy device;

FIG. 10 is a perspective of an exterior air fitting of an embodiment of the thermotherapy device;

FIG. 11 is an enlarged fragmentary perspective of the exterior air fitting on a closed kennel door and fluidly connected to an interior air fitting of the thermotherapy device mounted on a ducted pet bed inside the kennel;

FIG. 12 is an exploded perspective of an embodiment of a thermotherapy device and including a pet containment blanket;

FIG. 13 is an enlarged fragmentary cross section of a side portion of the thermotherapy device with the pet containment blanket installed;

FIG. 14 is a perspective of another ducted pet bed;

FIG. 15 is a fragmentary cross section of the pet bed of FIG. 14; and

FIG. 16 is a fragmentary perspective of another ducted pet bed.

Corresponding reference numbers indicate corresponding parts throughout the drawings.

### DETAILED DESCRIPTION

The inventors believe that the conventional thermotherapy devices for pet treatment could be improved. As explained above, when used in a kennel, a thermotherapy blanket requires a hose that runs from the blanket to the interior air fitting part. But in order to use the thermotherapy device, the door of the kennel must be opened to allow entry of an animal into the kennel and closed to contain the animal inside the kennel. Moreover, these door openings and closings must occur while the fitting and hoses are attached. This creates problems because the swinging motion of the kennel door tends to jostle the hose, which can cause the connection at one or both ends of the hose to break loose. Further, the hose can transfer forces to the blanket, displacing the blanket from the correct position on the kennel floor. The challenges compound because each time the hose is disconnected or the blanket is pulled out of position, the door must be reopened to rectify the issue. But each time the door is opened, it must be closed again before thermotherapy can begin, leading to further jostling of the hose and blanket.

Another challenge with thermotherapy blankets for pet use is that the blankets are inherently flexible and lightweight. There is no good way to secure a blanket in place, and so an animal will often inadvertently move a blanket out of position as the animal moves around to get comfortable in the kennel. When this occurs inside a kennel, the kennel must be reopened and the blanket must be repositioned, which starts the cycle of challenges related to deploying the thermotherapy blanket inside the kennel over again.

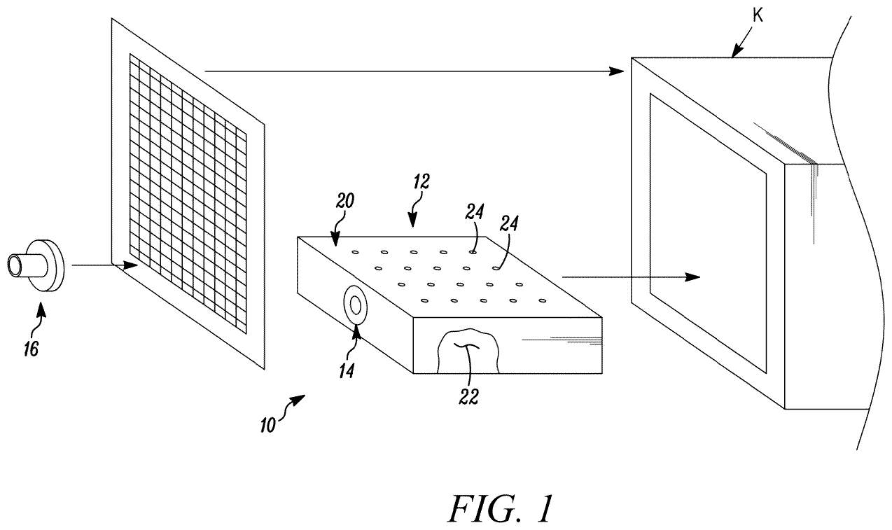

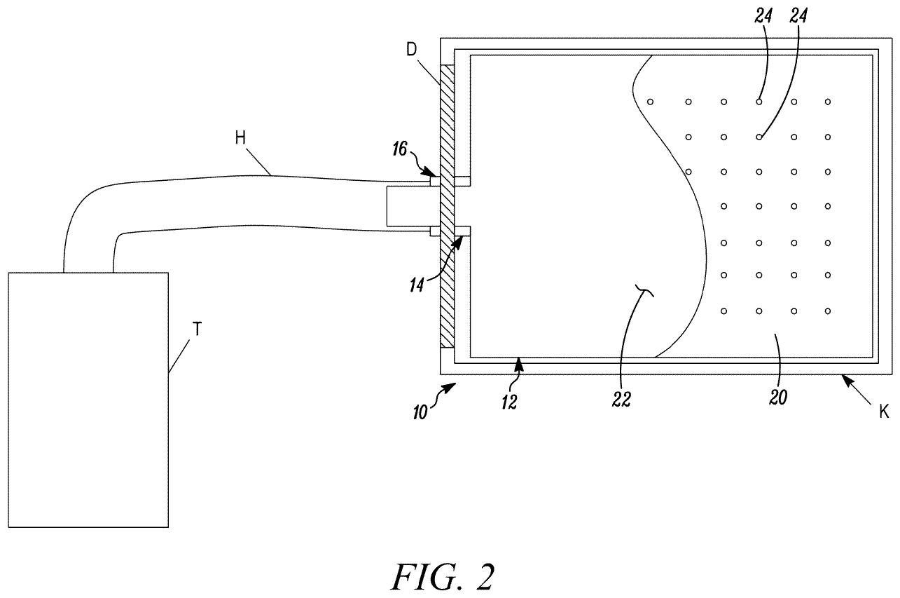

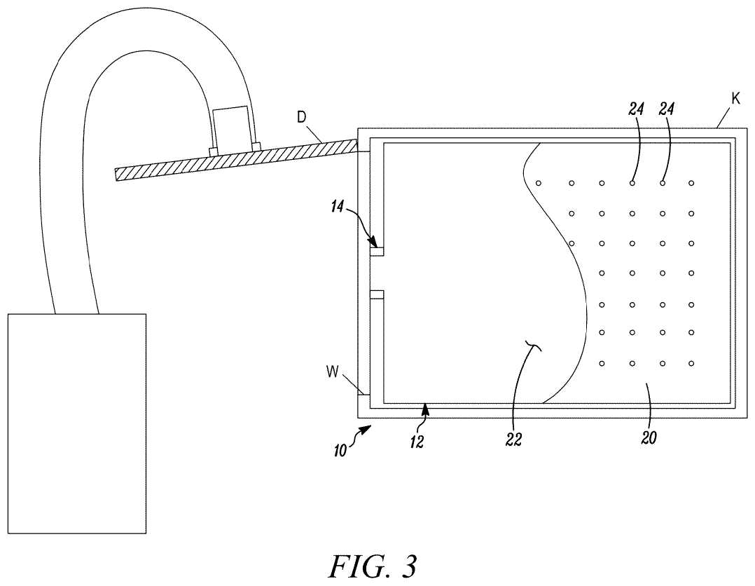

Referring to FIGS. 1-3, one embodiment of a thermotherapy device in accordance with the present disclosure is generally indicted at reference number 10. The thermotherapy device 10 broadly comprises a ducted pet bed 12, an interior air fitting 14 (which may also be called a bed-mounted air fitting when the thermotherapy device is used outside a kennel), and in some embodiments, an exterior air fitting 16. In the illustrated embodiment, the ducted pet bed 12 is sized and arranged for being positioned inside the kennel K. In will be understood, however, that the thermotherapy device can be used outside a kennel in certain embodiments. The ducted pet bed 12 comprises support sheet 20 for supporting a pet. The ducted pet bed 12 further comprises a plenum 22 below the support sheet 20 (broadly, the space below the support sheet can be considered an air flow space). The support sheet 20 defines a vent 24 in fluid communication with the plenum 22 so that air moving in the plenum is passable toward a pet supported on the support sheet. As can be seen in FIGS. 2 and 3, the ducted pet bed 12 has a perimeter footprint corresponding to an interior footprint of the kennel K such that side walls of the kennel hold the ducted pet bed at a stationary position on the kennel floor. As will be explained in further detail below, in one or more embodiments, the ducted pet bed 12 can have a rigid perimeter construction so that the ducted pet bed 12 can hold the interior air fitting 14 substantially stationary, e.g., stationary inside the kennel.

The interior air fitting 14 is supported on the ducted pet bed 12 in fluid communication with the plenum 22. As can be seen in FIG. 2, the ducted pet bed 12 and the interior air fitting 14 are arranged so that, when the ducted pet bed is positioned inside the kennel K, the interior air fitting is located adjacent a doorway W of the kennel to oppose an interior side of the door D when the door is in the closed position. The door D of the kennel K broadly comprises openings through which air is passable from outside the kennel to inside the kennel. For example, the kennel door D is typically a barred door with openings formed between the bars. In one or more embodiments, when the ducted pet bed 12 is positioned in the kennel K to hold the interior air fitting 14 in position adjacent the doorway W, the interior air fitting 14 is configured to make a fluid connection to the door D when the door is closed so that air directed through openings in the door flows into the interior air fitting. In certain embodiments, a large hole can be formed in the kennel door D so that a hose can be attached directly to the interior air fitting 14 through the hole in the door. The hose can also be directly attached to the interior air fitting 14 when the thermotherapy device 10 is used outside of a kennel K.

In the illustrated embodiment, the fluid connection between the interior air fitting 14 and the door D is made by direct contact between an inlet end portion of the interior air fitting and the door. For example, the interior air fitting 14 may comprise a compressible gasket at its inlet end portion that sealingly contacts the bars of the door D. In other embodiments, the fluid connection between the interior air fitting 14 and the door D can be made indirectly, e.g., by attaching a mating fluid coupling to the interior side of the door, which in turn mates with the interior air fitting on the ducted pet bed 12 when the door is closed. Regardless of whether the fluid connection between the bed-mounted air fitting and the door is made directly (e.g., via contact with the door) or indirectly (e.g., via mating with an additional fluid coupling mounted on the door), the thermotherapy device still comprises an interior air fitting configured to make a fluid connection to the door when the door is closed. In other words, in the present disclosure, unless otherwise noted, a fluid connection may be made between two parts by direct contact between the parts or by connection via one or more intervening fluid conveying structures.

As explained in further detail below, a temperature control device T is configured to blow a stream of temperature controlled air through openings in the door D. The interior air fitting 14 is configured to fluidly connect with the door D when the door is closed so that the interior air fitting 14 passes the temperature controlled air from a temperature control device T into the plenum 22. The temperature controlled air then moves in the plenum and passes through the vent 24 toward the pet supported on the support sheet 20.

As shown in FIG. 3, the interior air fitting 14 is configured to be separated from the door D each time the door moves away from the doorway W to an open position. Thus, in the illustrated embodiment, the fluid connection between the door D and the interior air fitting 14 is automatically disconnected each time the door is opened. But because of the way the ducted pet bed 12 positions the interior air fitting 14 at the operative position in the kennel K, the fluid connection is reestablished each time the door is closed. Moreover, the repeated fluid connection between the interior air fitting 14 and the door D is achieved without using flexible conduits between the door and the interior air fitting or between the interior air fitting and the plenum 22. In other words, flexible hoses and conduits can be eliminated inside the kennel K using the thermotherapy device 10 of the present disclosure. The inventors believe this makes it much easier to use the thermotherapy device 10 in a kennel K than prior art thermotherapy blankets.

The exterior air fitting 16 is configured to mount on the exterior side of the kennel door D such that the exterior air fitting moves with the kennel door as the kennel door moves between the closed position (FIG. 2) and the open position (FIG. 3). In other words, the exterior air fitting 16 can move relative to the interior air fitting 14 as the door opens and closes. Further, the exterior air fitting 16 is configured to align with the interior air fitting 14 when the door D is in the closed position such that the exterior air fitting fluidly communicates with the interior air fitting through openings in the door. Thus, the exterior air fitting 16 (being fixed to the door) can selectively connect to and disconnect from the interior air fitting 14 (being stationary in the kennel) when the door D is closed and opened, respectively.

The exterior air fitting 16 is configured to connect to a hose (e.g., a flexible air hose) H, which in turn connects to a temperature control device T. During use the temperature control device T blows temperature controlled air (typically, heated air) into the hose H, and the hose conveys the temperature controlled air to the exterior air fitting 16. When the door D is closed, the air stream is directed through openings in the door to the interior air fitting 14. The interior air fitting 14, in turn, directs the air stream to flow into the plenum 22. Finally, the temperature controlled air is directed upward through the vent 24 to provide thermotherapy to the pet supported on the support sheet 20.

As explained above, in certain applications, the hose H can be connected directly to the interior air fitting 14 instead of using an intervening exterior air fitting 16.

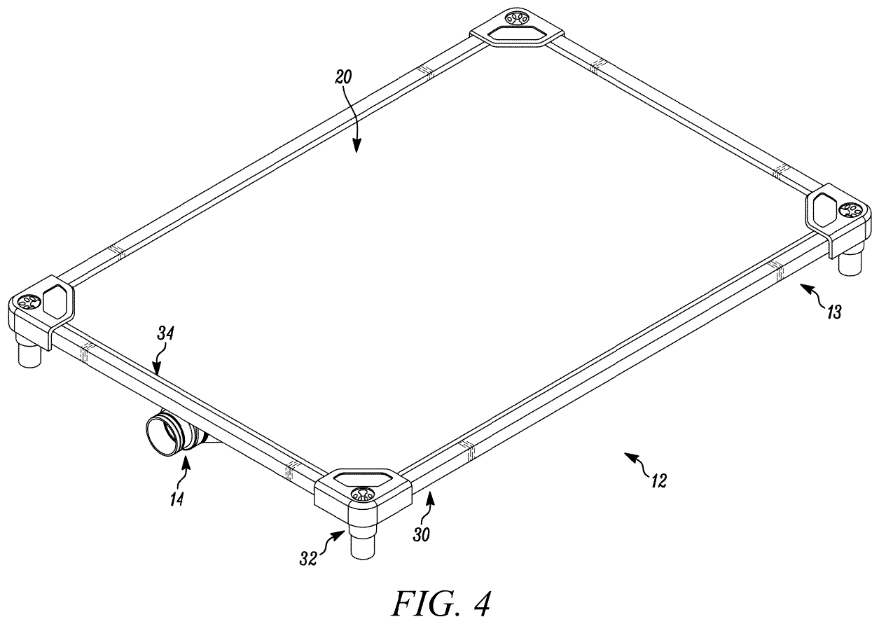

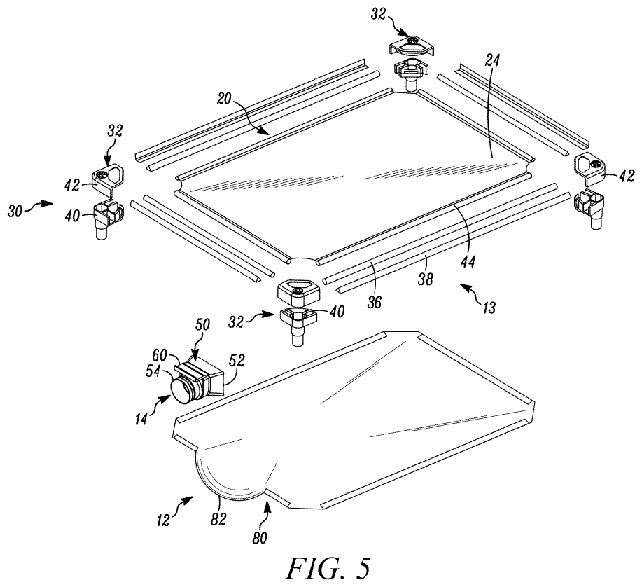

Referring now to FIGS. 4-5, an exemplary embodiment of the ducted pet bed 12 is shown in greater detail. In the illustrated embodiment, the ducted pet bed 12 comprises a cot 13 comprising a frame assembly 30 including legs 32 and a perimeter frame 34 supported on the legs. The legs 32 are broadly configured to support the perimeter frame 34 at an elevated position. The support sheet 24 is stretched over the perimeter frame 34 at the elevated position. That is, the frame assembly 30 is configured to hold the support sheet 20 in tension so that the support sheet extends generally in a plane that is spaced apart above the underlying support surface of the cot 12 (e.g., the kennel floor). Thus, the cot 12 is configured to support a pet on the support sheet 20 at an elevated position that is spaced apart above the underlying support surface of the cot.

As explained above, the support sheet 20 defines a vent 24 through which air is passable across the support sheet. In certain embodiments the vent 24 can be established as a function of the porosity of the material forming the support sheet 20, such that the vent is formed continuously along any portion of the support sheet in communication with the plenum 22. In other embodiments, the vent 24 can be defined by one or more discrete openings in the support sheet 20.

In the illustrated embodiment, the frame assembly 30 includes a plurality of perimeter frame members 36 defining a perimeter frame 34 and a plurality of perimeter shield members 38 defining a perimeter shield. The perimeter frame members 36 and perimeter shield members 38 extend between the legs 32 of the frame assembly 30. Together, the perimeter frame members 36 and the perimeter shield members 38 form four sides of a rectangular frame assembly 30. The legs 32 are positioned at the four corners of the cot 12 and are configured to interconnect the perimeter frame members 36 and perimeter shield members 38 to form the frame assembly 30. Each leg 32 forms a corner connector including a bottom portion 40 and a cap 42. The cap 42 is a separate component that can be selectively attached to the bottom portion 40 to connect the leg 32 to the respective perimeter frame and shield members 36, 38.

The frame assembly 30 can have the same basic design as the cot frame assembly described in U.S. Pat. No. 10,898,006, which is hereby incorporated by reference. Thus, as explained in U.S. Pat. No. 10,898,006, the bottom portion 40 of each leg 32 can further define two L-shaped sockets for matingly receiving end portions of two shield members 38 that extend from the leg in the assembled cot. As explained further in U.S. Pat. No. 10,898,006, the frame assembly 30 can be assembled by inserting the end portions of the shield members 38 into the sockets of the leg bottom portions 40 to form an intermediate frame subassembly. The frame members 36 can be installed in looped hems 44 of the support sheet 20, and then the end portions of the frame members can be loaded vertically into the open-top channels of the leg bottom portion 40 in the pre-assembled intermediate frame subassembly. Finally, the caps 42 can be installed on the leg bottom portions 40 to secure the frame assembly 30 together.

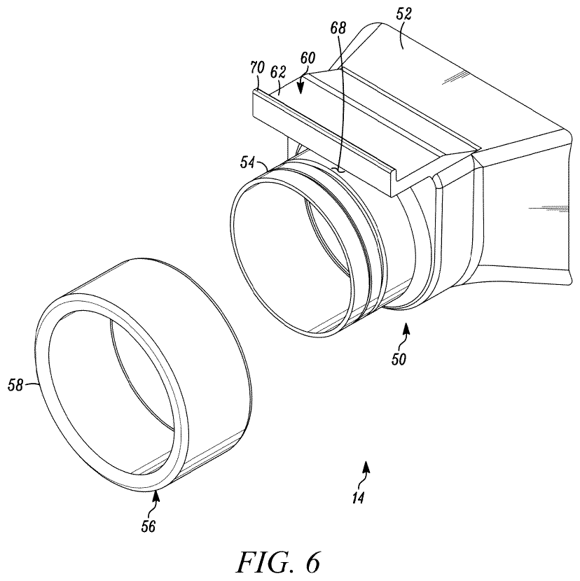

Referring to FIGS. 4-7, the interior air fitting 14 comprises an adapter body 50 comprising a plenum adapter 52 forming an outlet end portion and an inlet tube 54 opposite the plenum adapter. The adapter body 50 is hollow and defines a continuous air duct for directing an air stream that is imparted into the inlet tube 54 to flow out of the plenum adapter 52. In the illustrated embodiment, the inlet tube 54 is cylindrical and the plenum adapter has a rectangular perimeter with angled side walls. In one embodiment, the air fitting 14 is configured to fluidly connect to the kennel door D by direct contact. Thus, the illustrated interior air fitting 14 further comprises a compressible gasket 56 supported on the inlet tube 54. The compressible gasket 56 defines a contact surface 58 at which the interior air fitting 14 is configured for direct contact with the interior side of the kennel door D when the door is closed. In another embodiment, the compressible gasket is omitted and the inlet tube 54 connects directly to an air hose H.

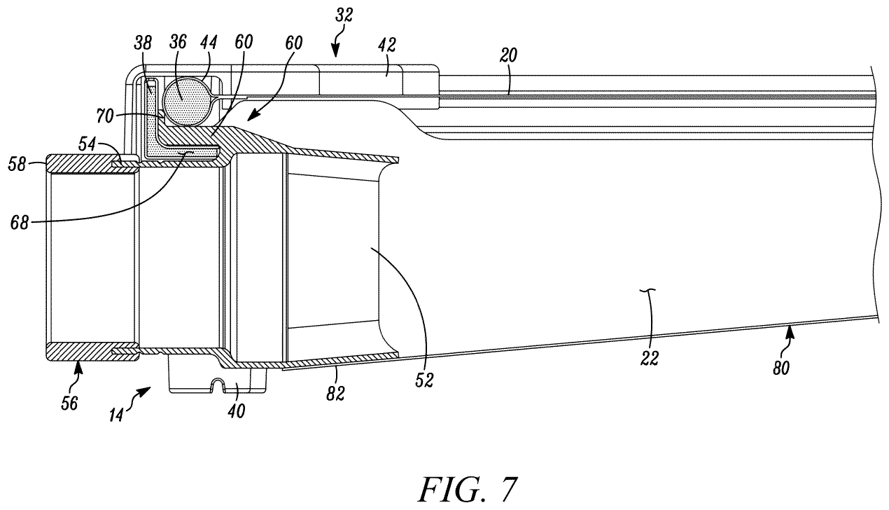

The illustrated adapter body 50 further comprises an integrated clip 60 configured to attach the interior air fitting 14 to the cot 12 (broadly, ducted pet bed) as described below. As shown in FIG. 7, the illustrated clip 60 is generally configured to clip onto a perimeter shield member 38 of the frame assembly 30 (broadly, the clip is configured to detachably couple to the frame assembly 30). The clip 60 comprises a cantilevered clip arm 62 that defines a clipping space 68 between the cantilevered segment and an upper wall portion of the adapter body 50. The interior air fitting 14 may be detachably coupled to the frame assembly 30 by locating at least a portion of the frame assembly within the clipping space 68. When the clip 60 is engaged, the clip 60 holds the interior air fitting 14 on the frame assembly 30 and may keep the interior air fitting from coming into contact with the underlying support surface of the cot 12 (e.g., the kennel floor). In the illustrated embodiment, the clip 60 is attached during cot assembly by inserting a horizontal portion of the L-shaped perimeter frame member 38 into the clipping space 68 and placing the frame member 36 on top of the clip arm 62 so that the clip arm is sandwiched between the perimeter shield member and the perimeter frame member 36. Thus, the clip 60 is partially located in a gap between a perimeter frame member 36 and the horizontal portion of a perimeter shield member 38. In one embodiment, the clip 60 further includes a lip 70 extending upwardly from the cantilevered segment 66. The lip 70 helps retain the fan on the frame assembly 12.

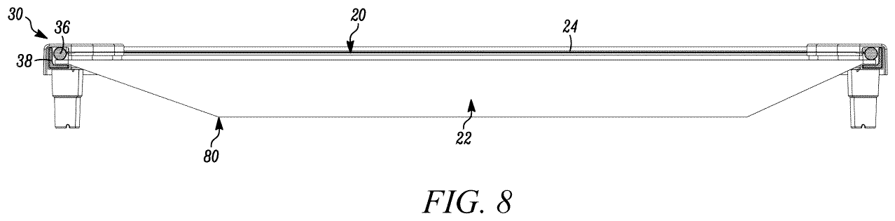

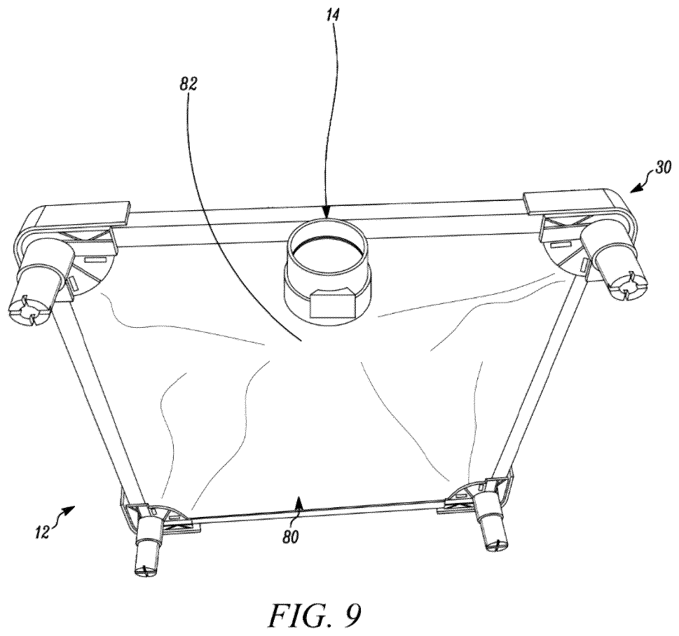

Referring to FIGS. 5 and 7-9, the cot 12 (broadly, the ducted pet bed) further comprises a ducting panel 80 below the support sheet 20. In general, the ducting panel 80 is configured to enclose the plenum 22 between the interior air fitting 14 and the vent 24 such that air directed into the plenum through the interior air fitting flows out of the plenum through the vent. In an embodiment, the ducting panel 80 is formed from flexible material that is less permeable than the material forming the support sheet 20.

In the illustrated embodiment, the ducting panel 80 is secured to the cot 12 at the frame assembly 30. More specifically, the ducting panel 80 has perimeter side edge margins that are sandwiched between the perimeter frames 38 and the perimeter shield members 36. In one or more embodiments, each perimeter side edge margin of the ducting panel 80 comprises panel material looped around a thin support bar that helps give the ducting panel its shape and retain the ducting panel on the frame assembly 30. The illustrated ducting panel 80 also comprises perimeter corner margins (which are devoid of support bars in the illustrated embodiment) sandwiched between the bottom portions 40 and the caps 42 of the legs 32.

The ducting panel 80 comprises a fitting region 82 adjacent one of the perimeter side edge margins. The fitting region 82 is configured to be stretched around the interior air fitting 14, specifically the plenum adapter 52. By connecting the perimeter edge margin of the ducting panel 80 to the frame assembly 30 and the plenum adapter 52 as described above, the ducting panel encloses the plenum 22 below the support sheet 20 so that substantially all air that is directed into the plenum through the interior air fitting 14 is directed to pass out of the plenum through the vent 24 in the support sheet 20. It will be understood that the way the ducting panel connects to the remainder of the ducted pet bed to define the plenum can vary from what is shown with departing from the scope of the disclosure. For example, it is contemplated that the ducting panel can be joined directly to the support sheet by one or more seams instead of having edge margins retained in the frame assembly.

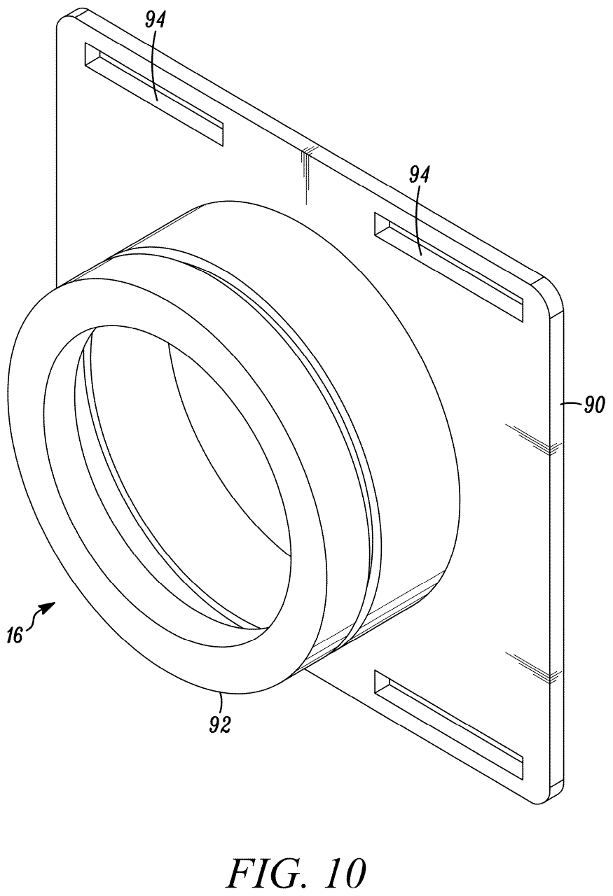

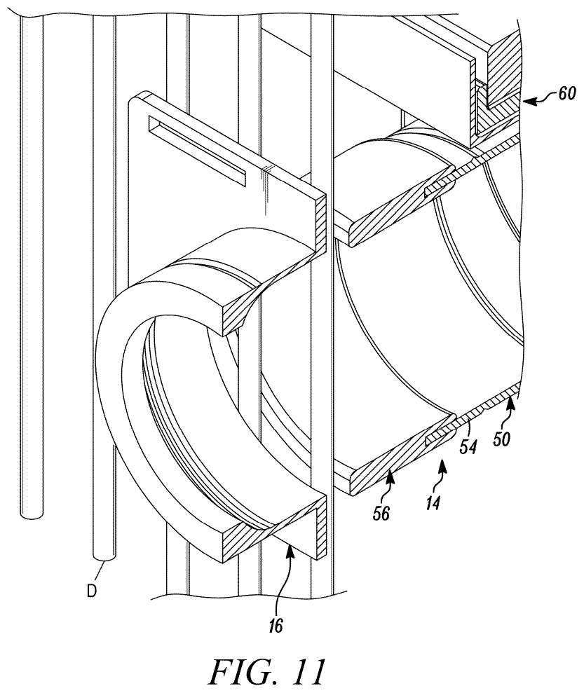

Referring to FIGS. 10-11, one suitable embodiment of an exterior air fitting 16 is shown in greater detail. In general, the exterior air fitting 16 is configured to be mounted on an exterior side of the kennel door D (see FIG. 11) and to align with the interior air fitting 14 when the door D is in the closed position such that the exterior air fitting fluidly communicates with the interior air fitting through the openings in the door. The illustrated exterior air fitting 16 comprises a door plate 90 configured to face the exterior side of the door D and a tube 92 extending outward from the door plate. The door plate 90 has fastener slots 94 through which removable fasteners (not shown) can be extended to secure the exterior air fitting 16 to the bars of the door D at a suitable position. It will be appreciated that the elongate slots allow for a degree of positional adjustability so that the exterior air fitting 16 can be mounted on the door D at a position that precisely aligns with the interior air fitting 14 where it is supported by the cot 12 inside the kennel K. The tube extends outward from the door plate 90 and is configured to connect to the flexible hose H that fluidly connects the exterior air fitting 16 to the temperature control device T.

Having described sufficient examples of thermotherapy devices in accordance with the present disclosure, an exemplary method of using the thermotherapy device 10 to treat a pet in a kennel K will now be described. Initially, a ducted pet bed 12 is constructed to have a footprint that corresponds to the interior footprint of the kennel K. For example, the shield members 38 of the desired size can be connected to the bottom portions 40 of four legs 32. Subsequently, the perimeter edge margins of the ducting panel 80 can be placed onto the assembly and the adapter body 50 can be clipped onto one of the shield members 38 so that the fitting region 82 of the ducting panel fits around the plenum adapter 52. Subsequently, the appropriate size frame members 36 can be inserted into the hems 44 of the support sheet 20 and loaded into the bottom portions 40 of the legs 32 from above. Last, the caps 42 may be secured to the lower leg portions 42 to complete the ducted pet bed 12.

After assembly of the ducted pet bed 12, it can be deployed along with the interior air fitting 14 inside the kennel K. The deployment positions the open inlet end of the interior air fitting 14 adjacent the doorway W of the kennel K. More specifically, the deployment positions the contact surface 58 of the gasket 56 to (sealingly) contact the interior side of the door D when the door is closed. Suitably, the deployed pet bed 12 is substantially immovable inside the kennel K (e.g., by virtue of the close fit of the rigid perimeter of the frame assembly 30 inside the side walls of the kennel) such that the position of the bed does not materially change, even as pets move around on the pet bed inside the kennel.

When the ducted pet bed 12 is well-positioned inside the kennel K, the user mounts the exterior air fitting 16 to the door at the proper position so that the exterior air fitting aligns with the interior air fitting 14 when the door D is in the closed position. Thus, whenever the door of the kennel K subsequently closes, the exterior air fitting fluidly connects to the interior air fitting through the openings in the door D.

After the thermotherapy device 10 has been set up in this manner, when a pet is in need of thermotherapy, the kennel door D is opened and the pet is brought into the kennel K through the doorway W while the door is in the open position, e.g., with the exterior air fitting 16 mounted on the door but fluidly disconnected from the interior air fitting 14. The pet then settles on the support sheet 20 of the ducted pet bed 12, which does not move substantially in relation to the kennel, even when the pet moves, unlike thermotherapy blankets in the prior art.

When the pet has been loaded onto the ducted pet bed 12 inside the kennel K, the door D is closed. This fluidly connects the exterior air fitting 16 supported on the door D to the interior air fitting 14 through one or more openings in the door. Notably, this fluid connection is established automatically simply by closing the door D. The user does not have to delicately handle flexible hoses or conduits that end up inside the kennel. There is no risk of hoses disconnecting as the door D swings open and closed. The user simply opens the door D, which momentarily disconnects the two fluid fittings 14, 16, and then closes the door which reconnects the two fluid fittings without any other effort.

Once the door D is closed, the user activates the temperature control device T to force temperature controlled air (typically, heated air) through the hose H, then the exterior air fitting 16. The temperature controlled air stream is directed from the exterior air fitting 16 into the interior air fitting 14 and then from the interior air fitting into the plenum 22 of the ducted pet bed 12. The temperature controlled air moving in the plenum 22 is directed through the vent 24 to apply thermotherapy to the pet resting on the support sheet.

It will be apparent to those skilled in the art that the method of using the thermotherapy device 10 will vary somewhat from the method described above when a hose H is connected directly to the interior air fitting 14 instead of making the fluid connection by contacting the door D with the interior air fitting.

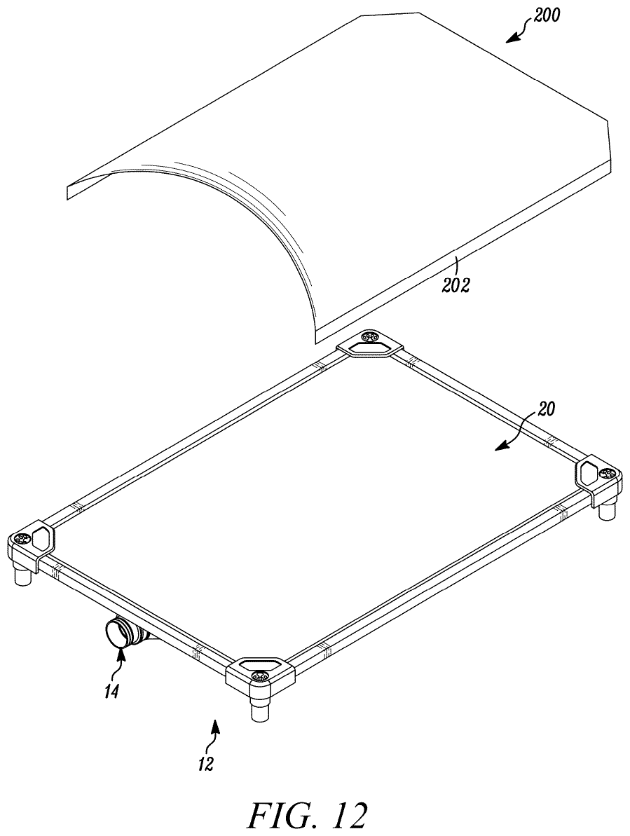

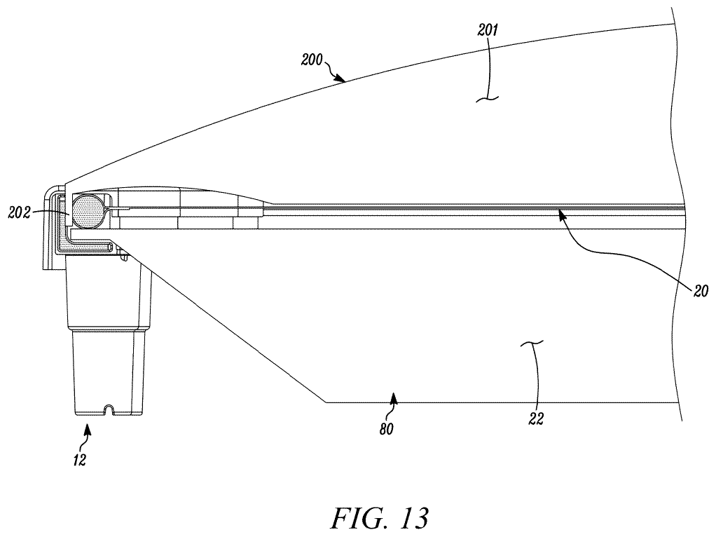

Referring to FIGS. 12 and 13, another configuration of the thermotherapy device 10 is shown. Here, an air containment blanket 200 is used in combination with ducted pet bed 12. The air containment blanket 200 comprises a fabric panel sized and arranged to be laid atop the ducted pet bed 12 to define a pet containment envelop between the support sheet 20 and the air containment blanket. The illustrated pet containment blanket 200 is configured to be connected to the ducted pet bed 12 by inserting perimeter edge margins 202 of the pet containment blanket in the spaces between the frame members 36 and the upright sections of the shield members 38. In one or more embodiments, the perimeter edge margins 202 contain rigid insertion members (e.g., elongate bars of more rigid material secured within looped hems of the pet containment blanket fabric). More particularly, three adjacent edge margins 202 of the pet containment blanket are tucked into three sides of the frame assembly 30 and the fourth side can be left unconnected from the frame assembly for pet access.

During use, the air containment blanket 200 can be installed before or after a pet is placed onto the ducted pet bed. When the temperature control device T blows air into the plenum 22 and the air flows through the vent 24, the pet containment blanket 200 forms an air flow guide that directs the temperature controlled air over the body of the pet. The air containment blanket 200 can be less air-permeable than the support sheet 20 in certain embodiments.

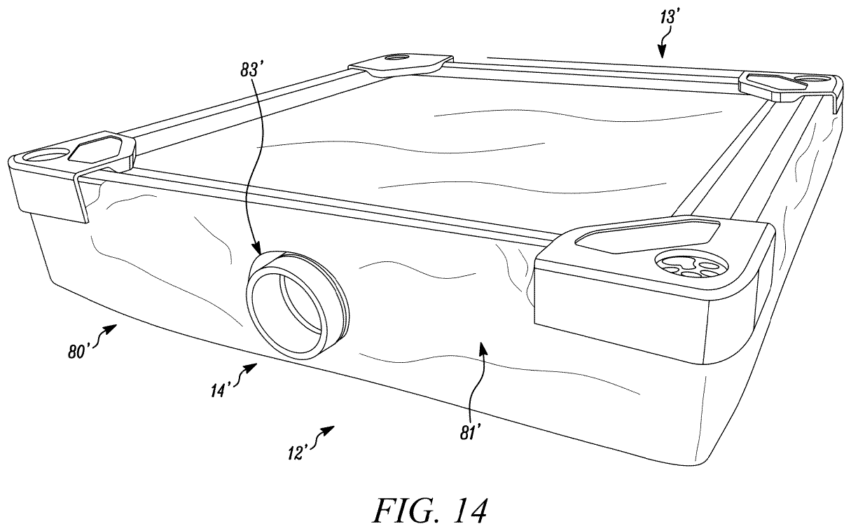

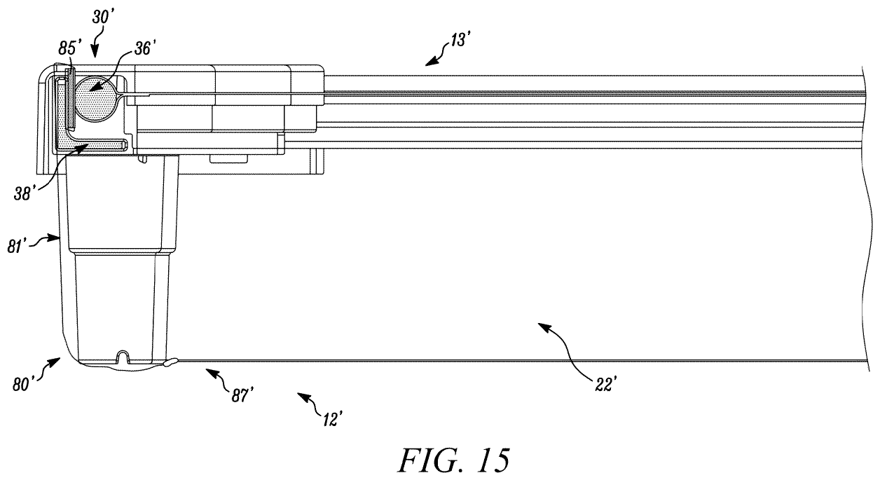

Referring to FIGS. 14-15, in another embodiment, of the ducted pet bed 12′, the ducting panel 80′ comprises a skirt portion 81′ configured to extend around the perimeter of the cot 13′ to define the plenum 22′. Besides the ducting panel 80′, all other features of a thermotherapy device incorporating the ducted pet bed 12′ can be the same as the thermotherapy device 10 described above.

The skirt portion 81′ comprises an upper edge margin configured to be releasably secured to the frame assembly 30′, e.g., by being pressed into the gaps between the frame members 36′ and the shield members 38′ as shown in FIG. 15. In one embodiment, the upper edge margin of the skirt portion 81′ includes four looped hems along each of four spaced apart side segments, and a flat strip 85′ of comparatively rigid material is received in each looped hem to aid in fitting the four upper edge margin segments into the gaps between the frame members 36′ and the shield members 38′. In another embodiment, separate shim strips (not shown) are used to secure the four upper edge margin segments of the skirt portion in the gaps. In certain embodiment, the skirt portion 81′ can include elastic material that constricts around the pet bed to retain the ducting panel 80′ on the pet bed. For example, the entire skirt portion 81′ can be formed from elastically stretchable material. A skirted ducting panel can also attach to a pet bed in other ways without departing from the scope of the disclosure.

The skirt portion 81′ further comprises a fitting opening 83′ configured to align with the inlet of the interior air fitting so that air can enter the interior air fitting 14′ through the fitting opening in the ducting panel. In the illustrated example, the gasket 56 protrudes through the fitting opening 83′ when the ducting panel 80′ is installed.

In one or more embodiments, the skirted ducting panel 80′ is an open-bottom panel. For example, the ducting panel extends heightwise from the upper edge margin to a lower edge margin configured to extend in a loop circumscribing a bottom opening 87′ (FIG. 15). Suitably, the skirt 81′ is sized (heightwise) to cover the entire height of the four sides of the cot 13′. In certain embodiments, the skirt 81′ is sized so that the lower edge margin turns underneath the feet of the cot 13′ when the upper edge margin is secured to the frame assembly 30′. In the case of an open-bottom ducting panel 80′, the ducting panel defines the sides of the plenum 22′ and the floor of the kennel K will define the bottom of the plenum during use. It is also contemplated that a skirted ducting panel with a skirt portion that circumscribes the perimeter of the cot can have a closed bottom. In these embodiments, the skirted ducting panel includes a bottom portion connected to the lower edge margin of the skirt portion and configured to cover the floor of the kennel below the pet bed.

In an example embodiment, the skirted ducting panel 80′ is configured to be removable, cleanable, and reusable. For example, the skirted ducting panel 80′ can be removed by disconnecting the upper edge margin from the frame assembly 30′ and pulling the ducting panel off the cot 13′. In certain embodiments, the ducting panel 80′ can comprise a zipper or other releasable closure to aid in removing and installing the ducting panel on the perimeter of the pet bed. Once removed the ducting panel 80′ can be cleaned and reinstalled on the same cot or a different cot. Suitably, the ducting panel 80′ is formed from launderable (e.g., canvas) or wipeable (e.g., vinyl) panel material that relatively air impermeable.

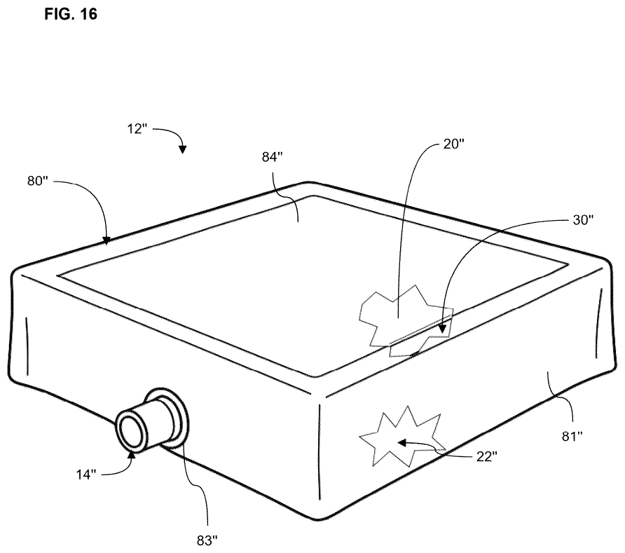

Referring to FIG. 16, another embodiment of the ducted pet bed 12″ comprises a covering panel in the form of a ducting panel 80″ that differs from the previously described embodiments. In this configuration, the ducting panel 80″ comprises both a skirt portion 81″ forming a perimeter portion and a topper portion 84″ that are pre-connected to form a single removable unit. The topper portion 84″ is configured to cover substantially the entire top surface of the frame assembly 30″ and support sheet 20″. The topper portion 84″ comprises air-permeable material configured to allow air from the air flow space below to pass upward through the topper portion 84″ toward a pet lying on the topper portion in the kennel.

The skirt portion 81″ forming the perimeter portion extends downward from the perimeter edges of the topper portion 84″, surrounding all four sides of the frame assembly 30″ and extending around the perimeter of the frame assembly 30″. In the illustrated example, the skirt portion 81″ forms a plenum 22″ (the space below the support sheet 20″ can more broadly be referred to as an air flow space) in similar fashion to how the skirt portion 81′ forms the plenum 22′ in FIGS. 14 and 15. In one or more embodiments, the perimeter portion comprises a skirt extending a full height of the frame assembly 30″ and enclosing a perimeter of the air flow space (but other perimeter portion configurations are also possible without departing from the scope of the disclosure). The skirt portion 81″ and topper portion 84″ are joined along their perimeter connection to form a continuous, integrated ducting panel 80″. In certain embodiments, the perimeter portion and the topper portion 84″ are formed from different materials, such as a less permeable material and/or an elastic material for the skirt portion 81″ and a more air-permeable material and/or less elastic material for the topper portion 84″.

The illustrated ducting panel 80″ encircles a plenum 22″ beneath the topper portion 84″ and support sheet 20″. The skirt portion 81″ defines the side boundaries of the plenum 22′ and the underlying support surface (such as a kennel floor) forming the lower boundary when the ducted pet bed is deployed.

The covering panel is broadly configured to be installed on the thermotherapy pet bed while the thermotherapy pet bed is in a veterinary kennel having an exterior air flow fitting mounted to an exterior side of a door of the veterinary kennel. The covering panel depicted here is shaped and arranged to permit a fluid communication connection to be automatically made from the exterior air flow fitting to the air flow space when the door of the veterinary kennel is closed such that temperature controlled air imparted through the exterior air fitting is directed into the air flow space and from the air flow space upward through the pet support surface and through the topper portion 84″ toward a pet lying on the topper portion in the kennel. In the illustrated example, the fluid communication is achieved via a fitting opening 83″ in the skirt portion 81″ configured to receive the interior air fitting 14″. Thus, in certain embodiments, the perimeter portion of a covering panel includes a front portion defining the fitting opening 83″ configured to receive the interior air fitting 14″. The fitting opening 83″ allows the interior air fitting to extend through the ducting panel while maintaining the enclosed nature of the plenum 22″.

The entire ducting panel 80″ can be removed from the frame assembly 30″ as a single unit for cleaning, maintenance, or replacement. The topper portion 84″ may include vents or may be formed from air-permeable material to allow temperature controlled air from the plenum 22″ to pass upward toward a pet supported on the ducting panel.

The integrated ducting panel 80″ may be secured to the frame assembly 30″ through various means. In one embodiment, the ducting panel 80″ is simply draped over the frame assembly 30″ and the interior air fitting 14″ is inserted into the fitting opening 83″. In other embodiments, additional fasteners such as elastic edges, snap connections, hook-and-loop fasteners are used. The removable ducting panel 80″ facilitates easy cleaning and maintenance while providing effective air distribution through the enclosed plenum 22″.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatuses, systems, and methods without departing from the scope of the invention, it is intended that all matter contained in the above description and shown in the accompanying drawings shall be interpreted as illustrative and not in a limiting sense.

1. A thermotherapy device for use in a kennel, the thermotherapy device comprising:
a ducted pet bed sized and arranged for being positioned inside the kennel, the ducted pet bed comprising a support sheet for supporting a pet and a plenum below the support sheet, the support sheet defining a vent in fluid communication with the plenum through which air moving in the plenum is passable toward the pet supported on the support sheet; and
an interior air fitting supported on the ducted pet bed in fluid communication with the plenum;
wherein the ducted pet bed and the interior air fitting are arranged so that, when the ducted pet bed is positioned inside the kennel the interior air fitting is located adjacent a doorway of the kennel to oppose an interior side of a door of the kennel when the door is in a closed position and to be separated from the door when the door moves away from the doorway to an open position,
wherein the interior air fitting is configured to make a fluid connection to the door when the door is in the closed position so that the interior air fitting passes temperature controlled air from a temperature control device flowing through one or more openings in the door into the plenum, whereby the temperature controlled air is caused to move into the plenum and pass through the vent toward the pet supported on the support sheet.

2. The thermotherapy device of claim 1, wherein the thermotherapy device is free of flexible conduit between the interior air fitting and the interior side of the door.

3. The thermotherapy device of claim 1, wherein the thermotherapy device is free of flexible conduit between the interior air fitting and the ducted pet bed.

4. The thermotherapy device of claim 1, wherein the interior air fitting has an inlet end portion defining a contact surface configured for direct contact with the interior side of the door and an outlet end portion connected to the plenum.

5. The thermotherapy device of claim 4, wherein the interior air fitting comprises an adapter body comprising a plenum adapter forming the outlet end portion and an inlet tube opposite the plenum adapter, the interior air fitting further comprising a compressible gasket supported on the inlet tube and defining the contact surface.

6. The thermotherapy device of claim 5, wherein the adapter body comprises an integrated clip configured to attach the interior air fitting to the ducted pet bed.

7. The thermotherapy device of claim 1, wherein the ducted pet bed has a perimeter footprint corresponding to an interior footprint of the kennel such that side walls of the kennel hold the ducted pet bed at a stationary position.

8. The thermotherapy device of claim 1, further comprising an exterior air fitting configured to be mounted on an exterior side of the door and to align with the interior air fitting when the door is in the closed position such that the exterior air fitting fluidly communicates with the interior air fitting through the one or more openings in the door.

9. The thermotherapy device of claim 8, wherein the exterior air fitting comprises a door plate configured to face the exterior side of the door and a tube extending outward from the door plate, the tube configured to connect to a flexible hose connected to an outlet of the temperature control device.

10. The thermotherapy device of claim 1, wherein the ducted pet bed comprises a cot comprising a frame assembly including legs and a perimeter frame supported on the legs, the legs configured to support the perimeter frame at an elevated position, the support sheet being stretched over the perimeter frame at the elevated position.

11. The thermotherapy device of claim 10, further comprising a ducting panel below the support sheet and configured to enclose at least perimeter sides of the plenum such that air directed into the plenum through the interior air fitting flows out of the plenum through the vent.

12. The thermotherapy device of claim 11, wherein the frame assembly further comprises a perimeter shield surrounding the perimeter frame and the support sheet.

13. The thermotherapy device of claim 12, wherein the interior air fitting comprises a clip configured to clip onto the perimeter shield.

14. The thermotherapy device of claim 13, wherein the ducting panel comprises perimeter side edge margins sandwiched between the perimeter frame and the perimeter shield.

15. The thermotherapy device of claim 14, wherein the ducting panel has a fitting region along one of the perimeter side edge margins, the fitting region being fitted around the interior air fitting.

16. The thermotherapy device of claim 15, wherein each leg comprises a bottom portion and a cap secured atop the bottom portion, wherein the ducting panel comprises perimeter corner margins, each of the perimeter corner margins sandwiched between the bottom portion and the cap of a respective leg.

17. The thermotherapy device of claim 11, wherein the cot has a perimeter and the ducting panel comprises a skirt portion extending around the perimeter of the cot.

18. The thermotherapy device of claim 17, wherein the ducting panel has an open bottom.

19. The thermotherapy device of claim 17, wherein the ducting panel has a closed bottom.

20. The thermotherapy device of claim 17, wherein the ducting panel is removable, cleanable, and reusable.

21-32. (canceled)
